(19) United States
(12) Patent Application Publication
(10) Pub. No.: US 2018/0110429 A1
(43) Pub. Date: Apr. 26, 2018

(54) Wearable Vital Signs Monitor

(71) Applicant: Massachusetts Institute of Technology, Cambridge, MA (US)

(72) Inventors: David Da He, Cambridge, MA (US); Eric S. Winokur, Danvers, MA (US); Charles G. Sodini, Belmont, MA (US)

(73) Assignee: Massachusetts Institute of Technology, Cambridge, MA (US)

(21) Appl. No.: 15/588,144

(22) Filed: May 5, 2017

Related U.S. Application Data
(63) Continuation of application No. 13/166,388, filed on Jun. 22, 2011, now abandoned.
(60) Provisional application No. 61/441,039, filed on Feb. 9, 2011.

(51) Int. Cl. A61B 5/024; A61B 5/0205; A61B 5/021; A61B 5/11; A61B 5/00; A61B 5/01; A61B 5/0402; A61B 5/029; A61B 5/1455; A61B 5/08

(52) U.S. Cl. CPC A61B 5/02438; A61B 5/02055; A61B 5/02125; A61B 5/11; A61B 5/6815; A61B 5/01; A61B 5/0816; A61B 5/029; A61B 5/02416; A61B 5/1102; A61B 5/14551; A61B 2560/0209; A61B 2562/0219; A61B 5/0402

(57) ABSTRACT

A method and monitor for monitoring vital signs. In one embodiment, the vital signs monitor includes a housing sized and shaped for fitting adjacent the ear of a wearer and an electronic module for measuring vital signs. The electronic module for measuring vital signs is located within the housing and includes a plurality of vital signs sensing modules in communication with a processor. The plurality of sensing modules includes at least two of the modules selected from the group of a ballistocardiographic (BCG) module, a photoplethysmographic (PPG) module, an accelerometer module, a temperature measurement module, and an electrocardiographic (ECG) module. In one embodiment, the processor calculates additional vital signs in response to signals from the plurality of vital signs sensing modules.

### FIELD OF INVENTION

This invention relates to the field of physiological monitors and more specifically to a wearable device for measuring vital signs.

### BACKGROUND

Monitoring vital signs is an important procedure in the caring for the elderly, sick and injured. Not only does the monitoring provide diagnostic clues as to the cause of the wearer's illness but also provides an advance warning if the wearer's condition is worsening.

In addition, healthy individuals frequently desire to measure certain vital signs as they exercise in order to track their physical condition both instantaneously and over time. Such monitors provide feedback to the user and help identify risks for certain diseases.

To monitor vital signs, multiple expensive specialized devices typically are used in a controlled setting such as a hospital. The bulk and costs of these devices make them inappropriate for home use. However, to reduce healthcare costs and to help the patient recover more quickly, frequently it is desired that a patient be moved from hospital care to homecare. Many times, this requires the renting of expensive equipment for long periods of time. In addition, sensors for measuring different vital signs at home are bulky and difficult to wear while the wearer is performing his or her ordinary functions.

What is needed is a device that will allow vital signs of a wearer to be monitored without the use of expensive bulky sensing devices and will allow the wearer to be able to perform their ordinary functions. The present invention addresses these issues.

### SUMMARY OF THE INVENTION

In one aspect, the invention relates to a vital signs monitor for wearing adjacent the ear. In one embodiment, the vital signs monitor includes a housing sized and shaped for fitting adjacent the ear of a wearer and an electronic module for measuring vital signs. In one embodiment, the electronic module for measuring vital signs is located within the housing and includes a plurality of vital signs sensing modules in communication with a processor. The plurality of sensing modules includes at least two of the modules selected from the group of a ballistocardiographic (BCG) module, a photoplethysmographic (PPG) module, an accelerometer module, a temperature measurement module, and an electrocardiographic (ECG) module. In one embodiment, the processor calculates additional vital signs in response to signals from the plurality of vital signs sensing modules. In another embodiment, the processor measures heart rate from the ECG, the BCG, or the PPG module. In yet another embodiment, the processor measures respiratory rate from the ECG, the BCG, or the PPG module. In still yet another embodiment, the processor determines orientation and motion in response to a signal from the accelerometer module. In one embodiment, the processor measures stroke volume in response to a signal from the BCG module. In another embodiment, the processor derives cardiac output in response to a signal from the BCG module. In yet another embodiment, the processor calculates blood pressure in response to signals from the ECG and the BCG modules. In still yet another embodiment, the processor calculates blood pressure in response to signals from the ECG and the PPG modules. In one embodiment, the processor calculates blood oxygenation in response to signals from the PPG module. In another embodiment, the processor measures temperature in response to a signal from the temperature measurement module. In another embodiment, the processor calculates the change in pre-ejection period in response to signals from the ECG and BCG modules.

In yet another embodiment, the electronic module further includes a display module for providing information to a user in response to measured and calculated vital signs. In one embodiment, the display module provides information to the user in response to measured and calculated vital signs that are out of acceptable range. In one embodiment, the display module provides auditory information. In another embodiment, the electronic module further comprises a memory module for saving recorded data. In yet another embodiment, the electronic module further comprises a wireless communication module for sending data to a base-station. In still another embodiment, the base-station provides feedback to a user in response to measured and calculated vital signs. In yet another embodiment, the base-station provides information to a user in response to measured and calculated vital signs that are out of acceptable range. In yet another embodiment, the base-station controls the operation of the electronic module based on measured and calculated vital signs. In still yet another embodiment, the processor performs, in response to one or more of the ECG signal, the BCG signal, the PPG signal, and the acceleration data, error detection for one or more of the heart rate, the respiratory rate, and the blood pressure. In another embodiment, the monitor further includes a switch the processor uses for turning on and off the BCG and the PPG modules in response to the ECG data, to reduce power consumption. In yet another embodiment, the monitor further includes a switch the processor uses for turning on and off the PPG module in response to the BCG data, to reduce power consumption. In still yet another embodiment, the monitor includes a switch the processor uses for turning on and off the ECG, the BCG, or the PPG module in response to accelerometer data so as to reduce power consumption. In still another embodiment, the monitor calculates the blood pressure using cross-correlation of either the ECG and the BCG signals, or the ECG and the PPG signals. In another embodiment, the monitor calculates the heart rate using cross-correlation of two of the ECG, the BCG, and the PPG signals.

In another aspect, the invention relates to a PPG monitoring device. In one embodiment, the PPG monitoring device includes a housing sized and shaped for fitting adjacent the ear of a wearer; and a PPG module located within the housing. The PPG module includes at least two light sources of different wavelengths positioned to transmit into the skin adjacent the ear of the wearer; at least one photodiode positioned to receive light reflected from the skin; and a first amplifier in communication with the photodiode and providing a first amplifier output signal. In another embodiment, the PPG monitoring device includes a demodulating circuit in communication with the first amplifier followed by a sample and hold circuit. In another embodiment, the PPG monitoring device includes third and fourth light sources having wavelengths differing from the other light sources. In another embodiment, the PPG monitoring device includes a high pass filter and a second amplifier in communication with the first amplifier. In still yet another embodiment, the PPG monitoring device includes a sample and hold circuit in communication with the second amplifier. In another embodiment of the PPG monitoring device, a difference amplifier is in communication with the first amplifier and subtracts a DC component and provides on an AC component sent to the second gain amplifier. In yet another embodiment, the PPG monitoring device further includes a low pass filter and a high pass filter in communication with first amplifier. In another embodiment, a bandpass filter, followed by a demodulator and a low pass filter are in communication with the first amplifier. In still yet another embodiment of the PPG monitoring device, the high pass, low pass and bandpass filters are implemented in software.

Another aspect of the invention relates to a BCG monitoring device. In one embodiment, the BCG monitoring device includes a housing sized and shaped for fitting adjacent the ear of a wearer and having two capacitive electrodes positioned in the mastoid region of the head of a wearer to sense head movements by transducing mechanical movements into electrical signals and a BCG module located within the housing. In another embodiment, the BCG monitor includes a differential signal amplifier having an output terminal and two input terminals, each input terminal in communication with a respective one of the capacitive electrodes and an analog-to-digital converter in communication with the output terminal of the differential signal amplifier. In yet another embodiment, the BCG monitoring device further includes a third electrode positioned at the mastoid region of the head of a wearer to reduce common mode interference signals.

In still yet another embodiment, the BCG monitoring device further includes a filter in communication with the output terminal of the differential signal amplifier to reduce interference signals. In one embodiment, the BCG monitoring device further includes an additional layer of electric shielding covering the two capacitive electrodes so as to reduce interference signals. In yet another embodiment, the BCG monitoring device further comprises of an accelerometer that senses head movements.

Another aspect of the invention relates to an ECG monitoring device. In one embodiment, the ECG monitoring device includes a housing sized and shaped for fitting adjacent the ear of a wearer; two dry or gel-based electrodes positioned at the mastoid region of the head of a wearer to sense ECG signals and an ECG module located within the housing. In one embodiment, the ECG module includes a differential signal amplifier having an output terminal and two input terminals, each input terminal in communication with a respective one of the dry or gel-based electrodes; and an analog-to-digital converter in communication with the output terminal of the differential signal amplifier. In another embodiment, the ECG monitoring device further includes a third electrode positioned in the mastoid region of the head of a wearer to reduce common-mode interference signals. In yet another embodiment, the ECG monitoring device further includes a filter in communication with the output terminal of the differential amplifier to reduce interference signals.

Yet another aspect of the invention relates to method for monitoring PPG of the user. In one embodiment, the method includes the steps of positioning a housing sized and shaped for fitting adjacent the ear of a wearer. The housing includes at least two light sources; a photodiode; a first amplifier in communication with the photodiode and providing an amplified output signal; and an analog-to-digital converter in communication with the amplified output signal; transmitting light from each of the light sources in an alternating manner to the skin of the mastoid region of the wearer; receiving, by the photodiode, the light reflected from the skin, tissue and bone of the mastoid region of the head of a wearer; amplifying, by the first amplifier, a signal generated by the photodiode in response to the light reflected from the skin, tissue and bone to generate an amplified output signal; and filtering the amplified output signal to reduce interference. In another embodiment of the PPG method, the signal filtering is performed in software.

Another aspect of the invention relates to a method for monitoring BCG. In one embodiment, the method includes positioning two capacitive electrodes at the mastoid region of the head of the wearer to sense head movements by transducing mechanical movements into electrical signals, and positioning a housing sized and shaped for fitting adjacent the ear of a user. In one embodiment, the housing includes a differential signal amplifier having an output terminal and two input terminals, each input terminal in electrical communication with a respective one of the two capacitive electrodes, and the output terminal in communication with an analog-to-digital converter. In one embodiment, the BCG method further includes the step of reducing common-mode interference signals by placing a dry electrode in the mastoid region of the head of a wearer. In another embodiment, the BCG method further includes filtering the output signal of the differential amplifier to reduce interference signals.

Yet another aspect of the invention relates to another method for monitoring BCG. In one embodiment, the method for measuring BCG includes the steps of positioning a housing containing an accelerometer that senses head movements and sized and shaped for fitting adjacent the ear of a user. In another embodiment, the BCG method further includes filtering the output of the accelerometer to reduce interference signals.

Another aspect of the invention relates to a method for monitoring an ECG. In one embodiment, the method includes the steps of positioning two electrodes at the mastoid region of the head of a wearer, positioning a housing containing a signal amplifier having an output terminal and two input terminals each in communication with a respective one of the electrodes and analog-to-digital converters in communication with the output of the amplifier, adjacent the ear of a user. In another embodiment, the ECG method further includes the step of positioning a third electrode in the mastoid region of the head of a wearer and using the third electrode to reduce common-mode interference signals. In another embodiment, the ECG method further includes the steps of filtering the output of the differential amplifier to reduce interference signals. In another embodiment, motion artifacts in the one or more of the ECG signal, the BCG signal, and the PPG signal are corrected using motion data from the accelerometer module.

### BRIEF DESCRIPTION OF THE DRAWINGS

The objects and features of the invention can be better understood with reference to the drawings described below. The drawings are not necessarily drawn to scale; emphasis is instead being placed on illustrating the principles of the invention. In the drawings, numerals are used to indicate specific parts throughout the various views. The drawings associated with the disclosure are addressed on an individual basis within the disclosure as they are introduced.

FIGS. 1a and b are diagrams of embodiments of the device of the invention located behind the ear of a patient;

FIG. 2 is a block diagram of an embodiment of the electronic modules of an embodiment of the system of the invention;

FIG. 3 is a block diagram of an embodiment of an ECG module of the invention;

FIG. 4 is a block diagram of an embodiment of a BCG module of the invention;

FIG. 5 is a block diagram of an embodiment of a PPG module of the invention;

FIG. 5A is a block diagram of another embodiment of a PPG module of the invention;

FIG. 6 is a flow diagram of the steps of one embodiment of a method for determining oxygen saturation in the blood of a user;

FIG. 7 is flow diagram of an embodiment of a method of cross correlating the heart rate waveforms to obtain heart rate measurements;

FIGS. 8A and 8B are flow diagrams of embodiments of methods of cross correlating the output of the ECG module and the output of the BCG module and PPG module respectively to obtain blood pressure;

FIG. 9 is a flow diagram of the steps of one embodiment of a method for error detection in the measurement of heart rate in a user;

FIG. 10 is a flow diagram of the steps of one embodiment of a method for error detection in the measurement of respiratory rate in a user;

FIGS. 11 (A,B,C) is a flow diagram of the steps of one embodiment of a method for error detection in the measurement of blood pressure in a user;

FIGS. 12-14 are flow diagrams of embodiments of methods of power saving; and

FIG. 15 is a block diagram of a method of removing motion artifacts from various waveforms.

### DETAILED DESCRIPTION

The following description refers to the accompanying drawings that illustrate certain embodiments of the invention. Other embodiments are possible and modifications may be made to the embodiments without departing from the spirit and scope of the invention. Therefore, the following detailed description is not meant to limit the invention. Rather, the scope of the invention is defined by the appended claims.

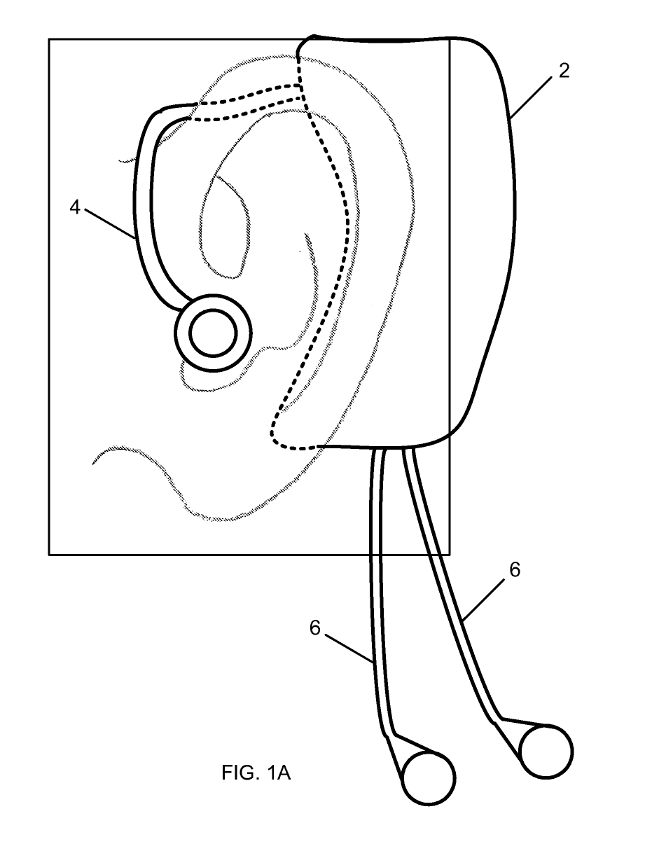

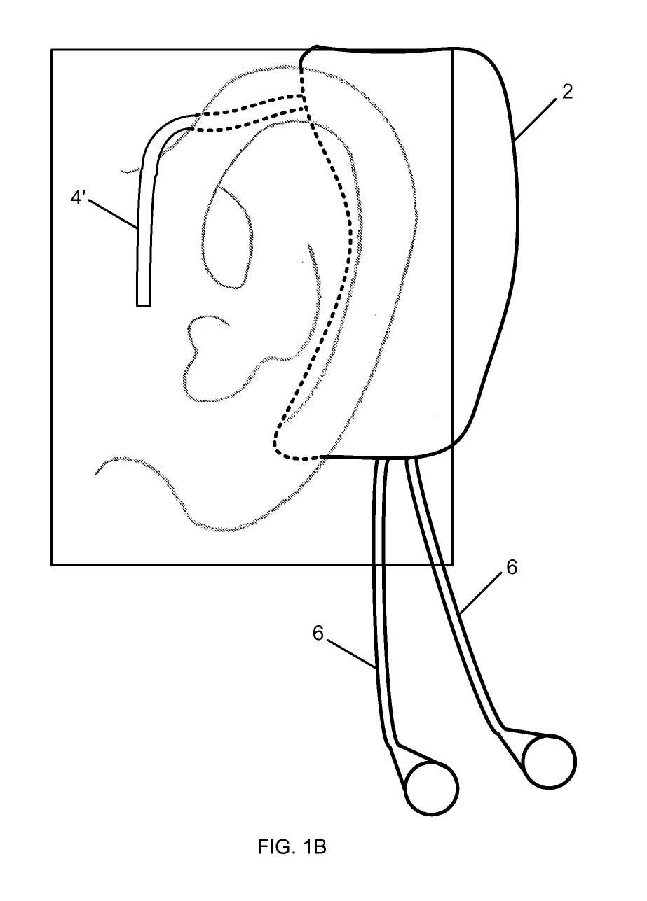

Referring to FIGS. 1a and b, in brief overview, two embodiments are depicted in which a device housing 2 fits behind the ear of a wearer, and is held in place by an earbud 4 located within the ear canal of the patient or by an earclip 4′ which fits over the ear of a wearer. In the embodiments shown, electrode leads 6 extend beyond the housing 2 and attach to electrodes mounted behind the ear of the wearer, near the wearer's mastoid. In another embodiment, the electrodes are built into the housing 2 and do not extend beyond the housing 2.

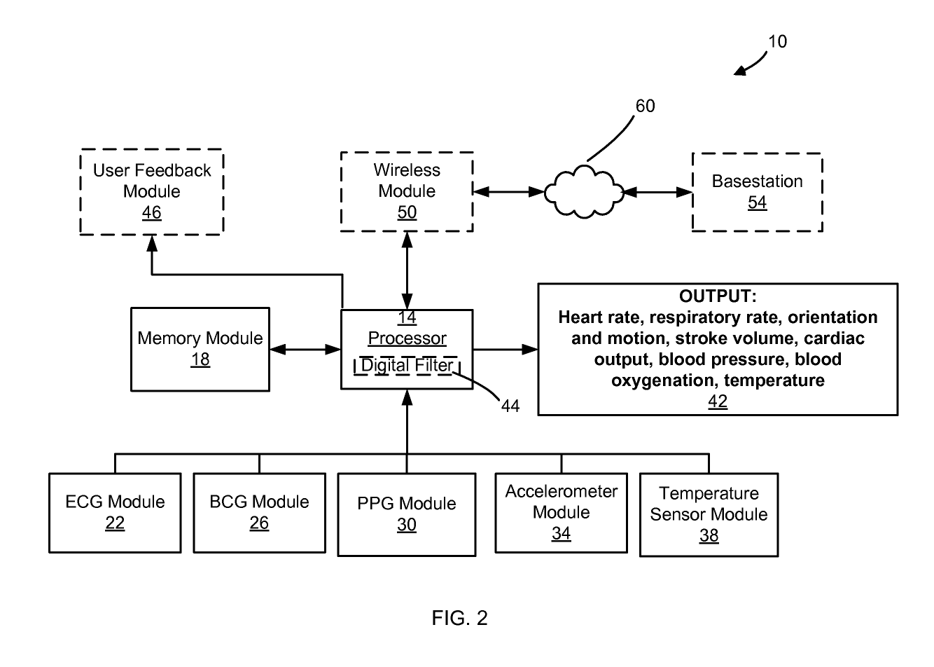

Referring to FIG. 2, the electronics of one embodiment of the system 10 includes a processor 14 in electrical communication with a memory 18 and two or more specialized data modules including, but not limited to, an electrocardiogram (ECG) module 22, a ballistocardiogram (BCG) module 26, photoplethysmographic (PPG) module 30, an accelerometer module 34 and a temperature sensor module 38. The processor 14 stores data from the modules in memory 18 and processes the data to derive additional vital signs. The processor 14 optionally includes digital filtering software 44 for use if the signals received from the modules are not prefiltered to reduce interference. The processor 14 is optionally in communication with a display module 42 (which may include or be an audible display), a module to provide feedback to the user 46, and a wireless module 50 (all shown in phantom). Additionally, if a wireless module is 50 used, data to the wireless module 50 may be transmitted directly to a base station 54 or communicated to the web 60 for communication to the base station 54.

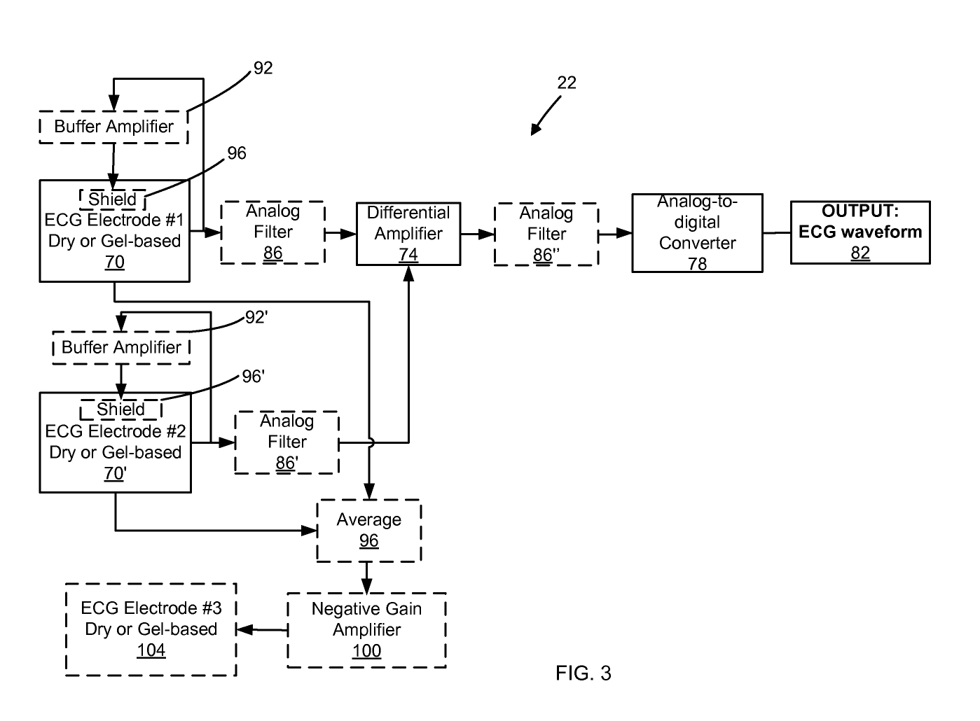

Considering each of the sensing modules individually, the ECG module 22 is shown in more detail in FIG. 3. In its simplest form, the ECG module includes an electrode 70 which may either be dry or gel based. The output of the electrode is one input to a differential amplifier 74. The output of a second electrode 70′ is the second input to the differential amplifier 74. The output of the differential amplifier 74 is in turn the input to an analog to digital (A/D) converter 78. The digitalized waveform output 82 of the A/D 78 is communicated to the processor 14 over a digital communication channel.

In other embodiments, an analog filter 86, 86′ may be placed in the circuit either immediately following the first and second electrodes 70, 70′or following (86″) the differential amplifier 74. The analog filter 86, 86′, 86″ is a notch filter to remove DC and powerline interference. In one embodiment, the outputs of the ECG electrodes 70, 70′ are inputs to respective buffer amplifiers 92, 92′, whose output terminals are connected to their respective active electrode shields 96, 96′ to reduce interference from the environment.

In one embodiment, the output of each ECG electrode is the input to a signal averager 96 whose output is a common-mode signal which is the input to a negative gain amplifier 100. The common-mode amplified output of the negative gain amplifier 100 is connected to an optional third dry or gel-based electrode 104 to reduce common-mode interference.

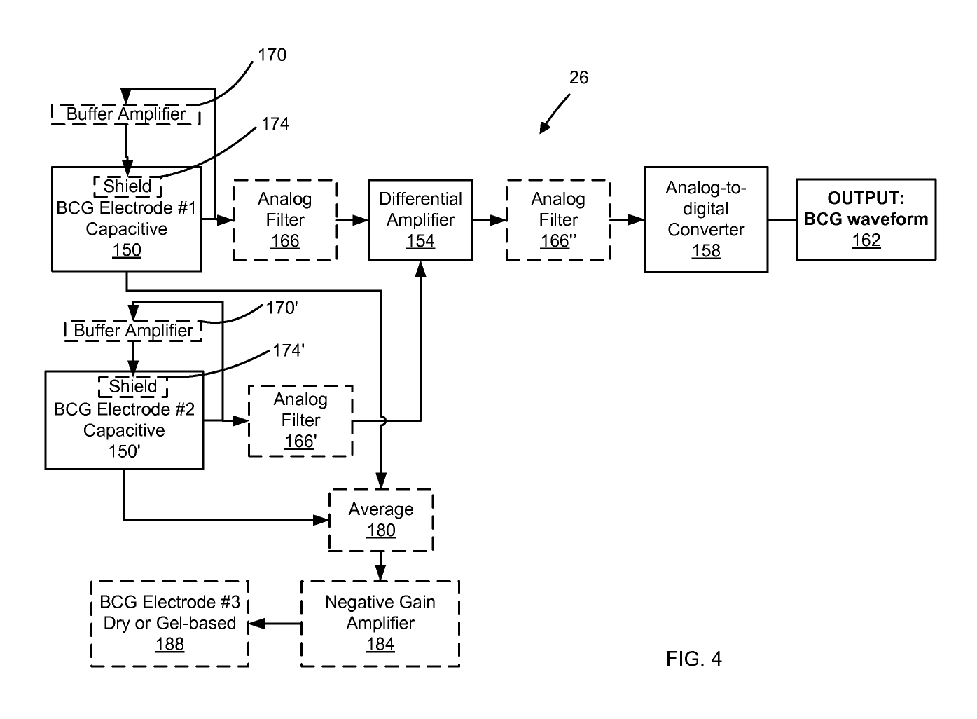

Similarly, one embodiment of the BCG module 26 is shown in FIG. 4. In this embodiment, two BCG electrodes 150, 150′ generate output signals which are the input signals to a differential amplifier 154 whose output is the input signal to an A/D converter 158. The digital output of the A/D 158 is transmitted to the processor 14 as a digitized digital BCG waveform 162. On some embodiments, an analog filter 166, 166′ is placed after each electrode 150, 150′or after (166″) the differential amplifier 154. In one embodiment, the output signals of the BCG electrodes 150, 150′ are input signals to respective buffer amplifiers 170, 170′, whose output terminals are connected to their respective active electrode shields 174, 174′.

In one embodiment, the output of each BCG electrode 150, 150′ is the input to an averager 180 whose output is the input to a negative gain amplifier 184. The output of the negative gain amplifier 184 is connected to a third dry or gel-based electrode 188 as discussed above to reduce interference.

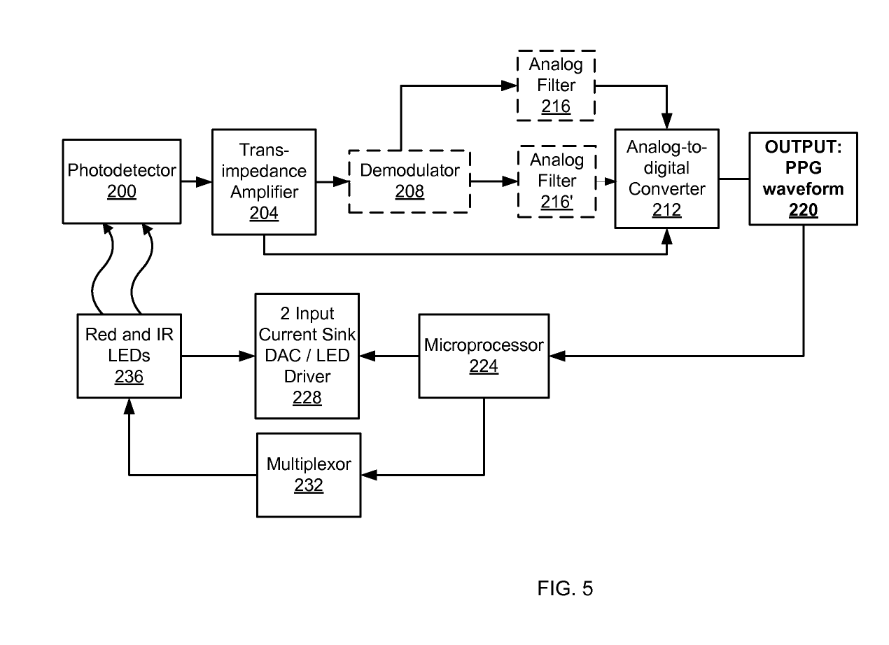

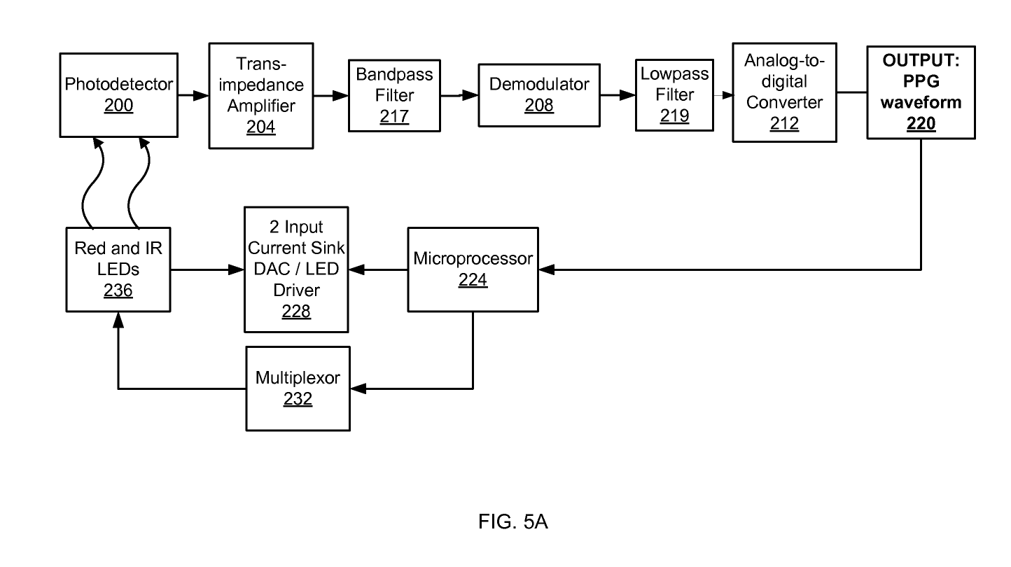

Referring to FIG. 5, an embodiment of a PPG module 30 includes a photodetector 200 whose output is an input to a transimpedance amplifier 204. The output of the transimpedance amplifier 204 is the input to an A/D converter 212 whose PPG waveform output is communicated to the processor 14. In one embodiment, the output of the transimpedance amplifier 204 is the input to a demodulator 208. The demodulator is used to separate the red and infra-red signals from an LED illuminator as described below, so that they may be filtered separately. The two output signals of the demodulator are input signals to two respective analog filters 216, 216′ and the output signals of the analog filters 216, 216′ are inputs to an A/D converter 212. Again the PPG waveform output 220 of the A/D converter 212 is communicated to the processor 224.

Referring to FIG. 5A, in another embodiment, the output of the transimpedance amplifier 204 is an input signal to a bandpass analog filter 217. The output of the bandpass analog filter 216 is the input to a demodulator 208, and the demodulator 208 output is in turn the input to lowpass analog filter 219. The output signal of the lowpass analog filter 219 is an input to an A/D converter 212. Again the PPG waveform output 220 of the A/D converter 212 is communicated to the processor 224.

In the case where the demodulator 208 is not used, the output is taken directly from the first amplifier, and is transmitted to the processor which filters and demodulates the signal in software.

The microprocessor 224 also provides output control signals to a multiplexor 232 to turn on and off red and infra-red light emitting diodes 236. The microprocessor 224 also provides control signals to an LED driver to control current through the red and IR LEDs.

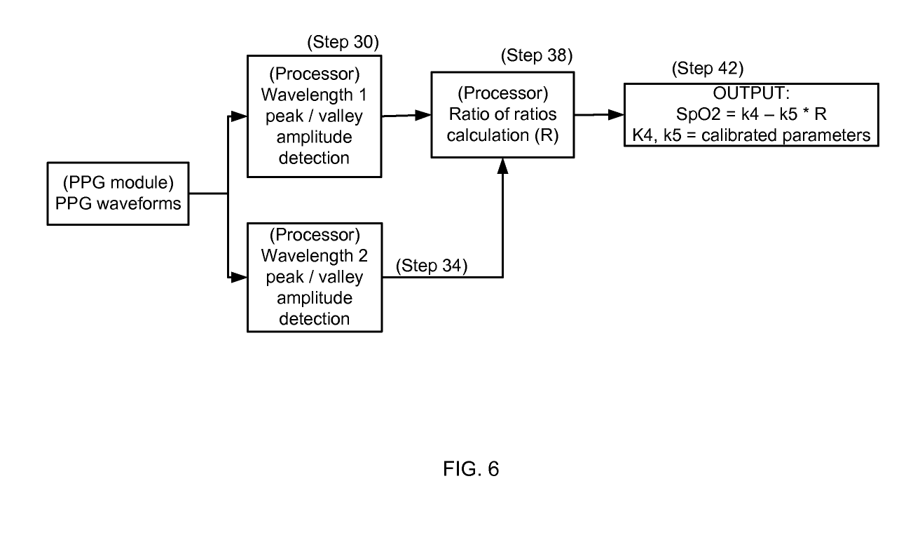

The user's oxygenation (FIG. 6) is measured by taking the PPG waveform signals from the PPG module 30, and detecting the ratio of the amplitudes of the peak/valley at each wavelength (steps 30, 34). These two ratios are then processed (Step 38) to obtain a ratio (R) of the two ratios. The oxygen saturation is then calculated (Step 42) as equal to a calibration constant (k4) minus the quantity of [(R) times a second calibration parameter (k5)].

The calibrations constants (k4) and (k5) in one embodiment are derived in a clinic. While wearing the device, the wearer is fitted with an indwelling arterial cannula, which is placed in the radial artery. A sample of blood is taken and analyzed with a CO-oximeter (gold standard blood oxygenation measurement device) to determine the wearer's level of functional hemoglobin. Once a high level of functional hemoglobin is verified, the wearer is fitted with one or more oximeter probes. The wearer breathes an oxygen/gas mixture. This mixture is at first rich in oxygen so as to ensure the wearer's blood oxygenation is 100%. Oxygen is then progressively decreased from the mixture and once a stable oximeter reading is taken at each level, a blood sample is taken to compare the R ratio generated from the oximeter and the actually blood oxygenation. The oximeter is then calibrated by using a best fit curve for the R ratios and blood oxygenation using constants k4 and k5

The processor 14, upon receipt of signals from the various modules, processes those signals to determine vital signs. For example, the heart rate of a user may be determined by the processor 14 from the signals from the ECG module 22, the BCG module 26 and/or the PPG module 30. In each case, the processor 14 uses peak detection to determine the peak in the signal from the ECG module 22, the signal from the BCG module 26 or the signal from PPG module 30, as the case may be. The processor 14 then divides sixty seconds by the time period between the peaks to obtain the heart rate.

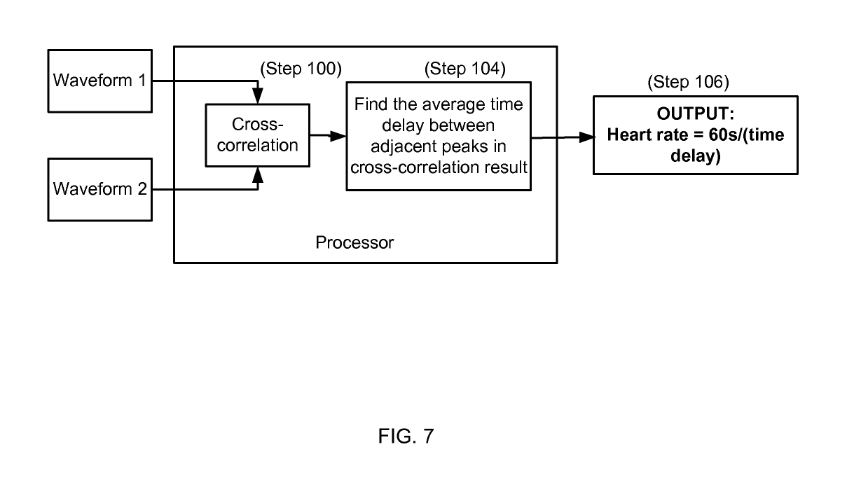

Referring to FIG. 7, in another embodiment the heart rate is calculated using cross-correlation of two of the ECG, the BCG, and the PPG waveforms in the time domain. In this embodiment, the two waveforms are cross correlated (Step 100). The average time between adjacent peaks in the cross-correlation result is measured (Step 104) and the heart rate is calculated as sixty seconds divided by the average time between adjacent peaks (Step 106). The user's respiratory rate can be determined by the processor 14 from signals from the ECG module 22, the BCG module 26, and the PPG module 30 by detecting the number of oscillations of the envelope of the signal from the given module in a one minute window.

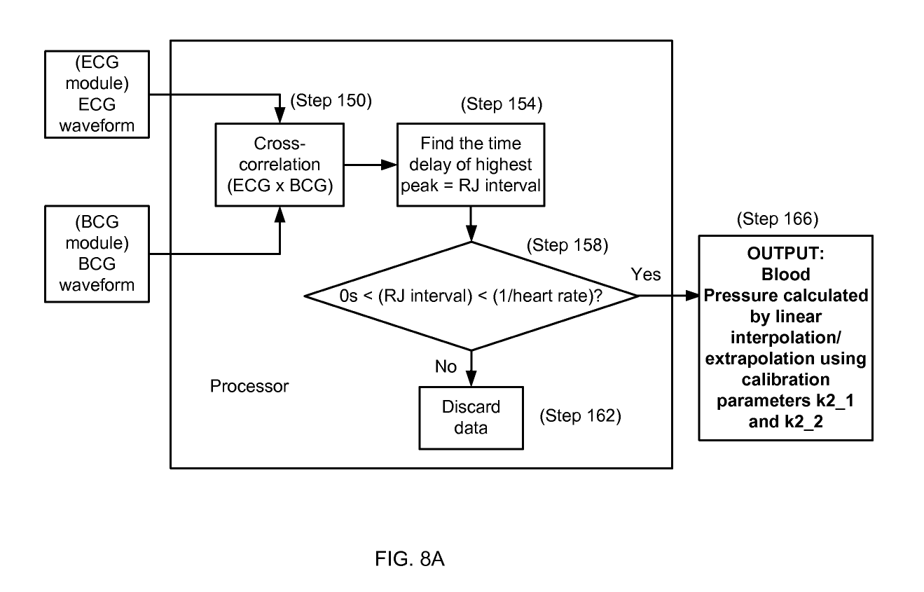

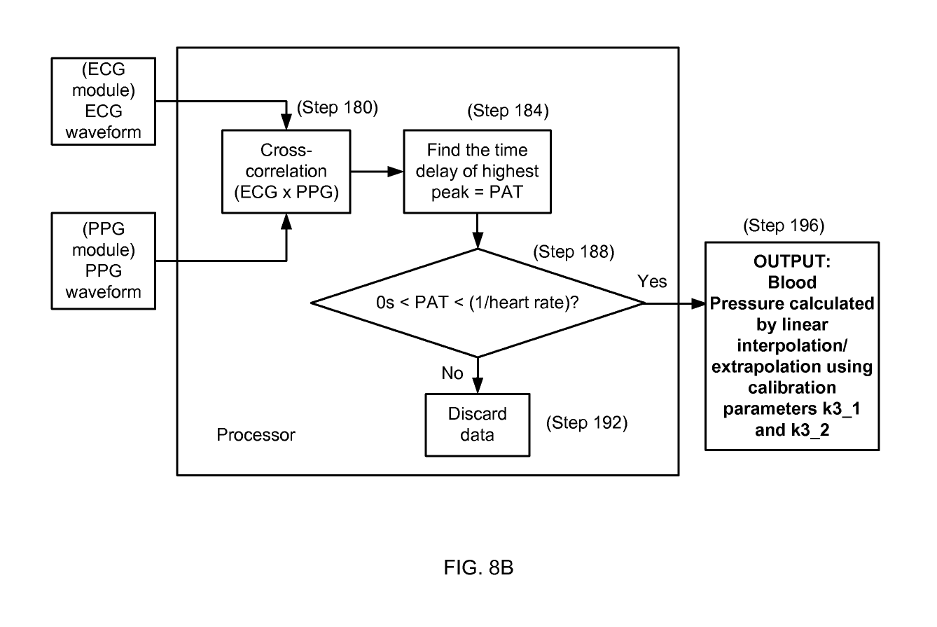

Referring to FIG. 8A, the blood pressure of a user can be calculated by cross-correlating (Step 150) the ECG and the BCG waveforms and determining the time delay for the highest peak (Step 154). Defining this time delay as the RJ Interval, the processor 14 then determines if the RJ Interval is greater than zero and less than one divided by the heart rate (Step 158). If this condition is not met the data is simply discarded (Step 162). If the condition is met, the RJ interval is recorded. Blood pressure is calculated by linear interpolation/extrapolation using calibration parameters k2_1 and k2_2.

Alternatively, (FIG. 8B) the user's blood pressure can be calculated by cross-correlating (Step 180) the ECG and the PPG waveforms and determining the time delay for the highest peak (Step 184). Defining this time delay as the Pulse Arrival Time (PAT), the processor 14 then determines if the Pulse Arrival Time is greater than zero and less than one divided by the heart rate (Step 188). If this condition is not met the data is simply discarded (Step 192). If the condition is met, the PAT is recorded. Blood pressure is calculated by linear interpolation/extrapolation using calibration parameters k3_1 and k3_2.

To determine the calibration constants (k2 and k3), the wearer's systolic blood pressure (SBP) is measured using a standard cuffed blood pressure measurement method and this is entered into the device as SBP-1. Next, the recorded RJ interval (RJ-1) and Pulse Arrival Time PAT-1 are also recorded as described above. Next, another systolic blood pressure measurement is made SBP-2 using the cuffed BP method and SBP-2 is entered into device. SBP-2 must differ by 10 mm Hg from SBP-1. If SBP-2 differs from SBP-1 as required, a second RJ interval (RJ-2) and Pulse Arrival Time PAT-2 are also measured.

This data is fit to an RJ interval linear model using SBP-1, RJ-1, SBP-2, and RJ-2.The slope (k2_1) and offset (k2_2) parameters are then measured. Next, the Pulse Arrival Time is fit to a linear model using SBP-1, PAT-1, SBP-2, and PAT-2. Again, the slope (k3_1) and offset (k3_2) parameters are measured. Using this data, all future measured RJ intervals are mapped to SBP by linear interpolation/extrapolation using k2_1 and k2_2 and all future measured Pulse Arrival Times are mapped to SBP by linear interpolation/extrapolation using k3_1 and k3_2.

The heart's pre-ejection period (PEP) is defined as the delay from the depolarization of the heart's septal muscle to the opening of the aortic valve. PEP can be used to determine the heart's contractility and muscle health. The relative change in the RJ interval obtained from ECG and BCG can be used to approximate the relative change in the PEP.

The relative stroke volume of a patient is also derived by the processor 14 from the waveform from the BCG module 26. The processor 14 detects a peak in the BCG waveform and measures the amplitude of that peak. The stroke volume of the wearer at rest, as determined by the accelerometer value is then set equal to the peak amplitude in the BCG waveform. All other stroke volumes, not at rest, are reported relative to this resting stroke volume. The patient's relative cardiac output is derived from the relative stroke volume of the user (as described above) and the heart rate of the user. The relative cardiac output is equal to the relative stroke volume multiplied by the heart rate.

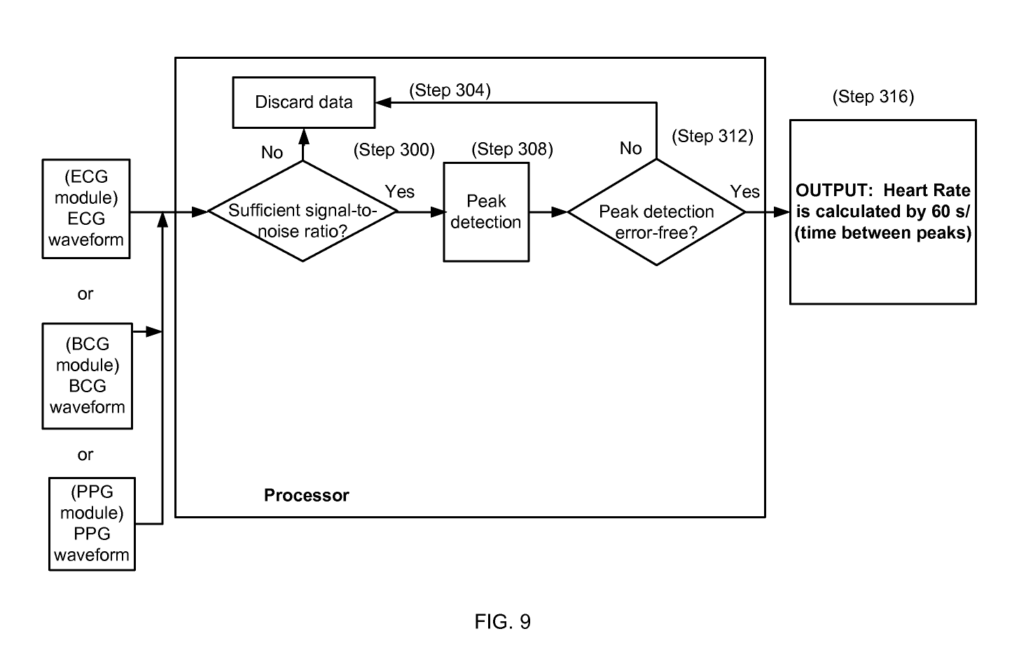

Referring to FIG. 9, to determine if there is an error in the heart rate measurement, the processor 14 obtains waveform data for a fixed time window, from the source of the heart rate signal, such as the ECG module 22, the BCG module 26 or the PPG module 30. The processor 14 then determines if the signal to noise ratio (S/N) is sufficient (Step 300) and if not the data is discarded (Step 304) and additional data collected. In one embodiment, the S/N ratio is deemed sufficient if the signal level is substantially 1.5 times the noise. If the S/N ratio is sufficient, peak detection (Step 308) is performed on the waveform. In one embodiment, if that peak detection is not substantially error free, because there are too many or too few peaks detected compared to previous time windows (Step 312), the data is also discarded (Step 304) and additional data is collected. If the peak detection is substantially error free, the heart rate calculation is then made (Step 316).

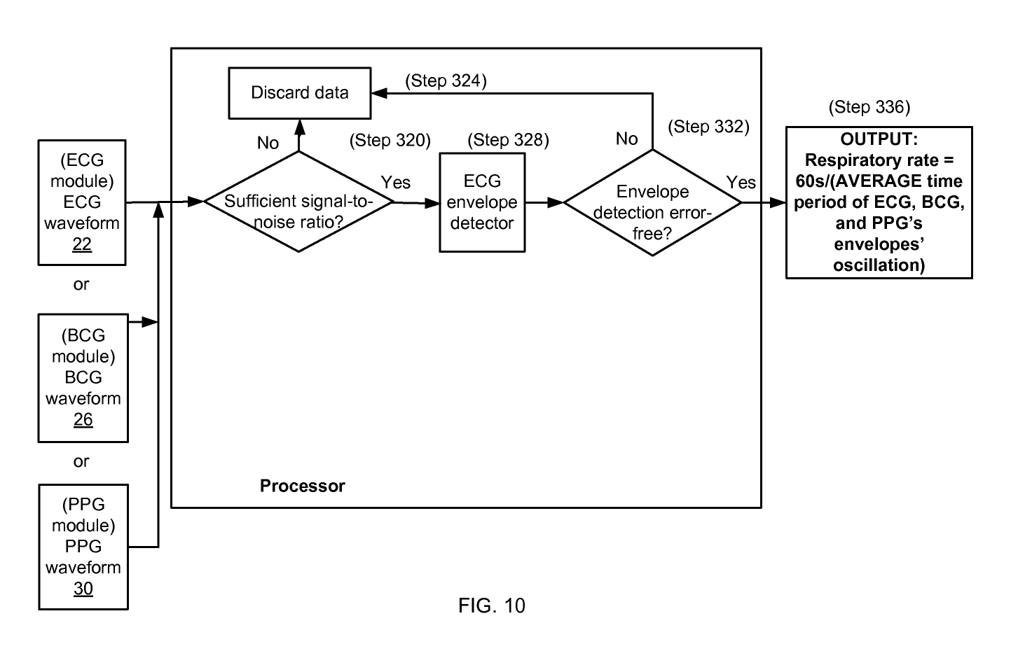

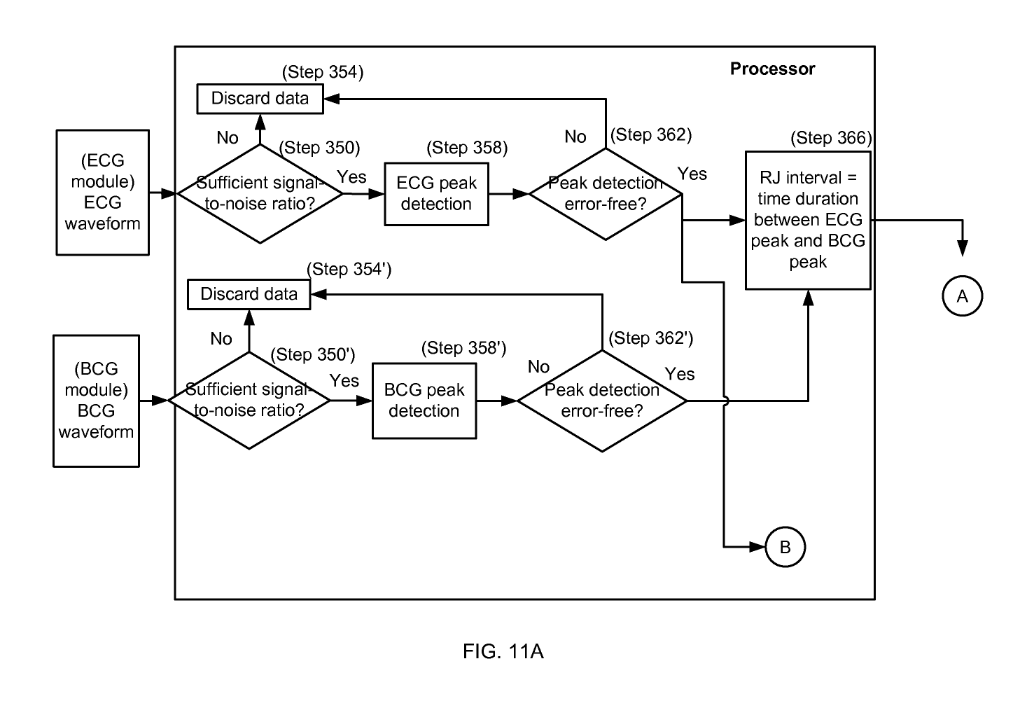

Similarly, referring to FIG. 10, to determine if there is an error in the respiratory rate measurement, the processor 14 obtains waveform data from the source of the respiratory rate signal, such as the ECG module 22, the BCG module 26 or the PPG module 30. The processor 14 then determines if the signal to noise (S/N) ratio is sufficient (Step 320), as discussed above, and if not the data is discarded (Step 324) and additional data collected. If the S/N ratio is sufficient, envelope detection (Step 328) is performed on the waveform. If the envelope detection is not substantially error free (Step 332), as discussed above, the data is discarded (Step 324) and additional data is collected. If the envelope detection is substantially error free, the respiratory rate calculation is then made (Step 336).

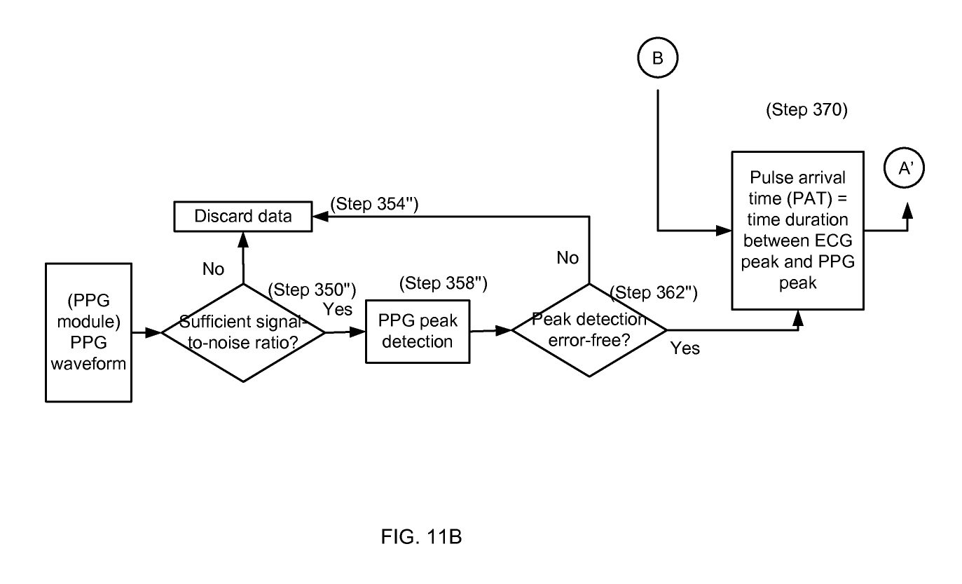

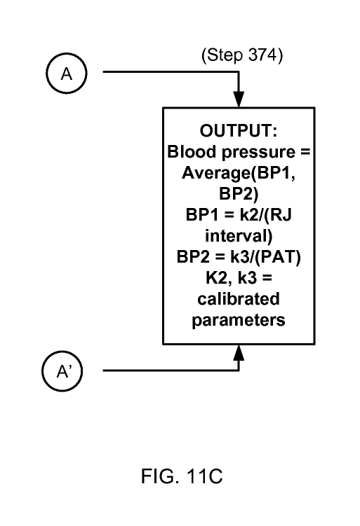

Referring to FIGS. 11 (A,B,C), to determine if there is an error in the blood pressure measurement, the processor 14 obtains waveform data for the source of a heart rate signal, such as the ECG module 22, the BCG module 26 and the PPG module 30. The processor 14 then determines if there is the signal to noise (S/N) ratio is sufficient (Step 350, 350′, 350″) and if not the data is discarded (Step 354, 354′, 354″) and additional data collected. If the S/N ratio is sufficient, peak detection (Step 358, 358′ 358″) is performed on the waveform. If that peak detection is not substantially error free (Step 362, 362′, 362″) the data is discarded (Step 304) and additional data is collected. If the peak detection is substantially error free, the peak detection information from the ECG module 22 is used by the processor 14 as an input to both of the RJ Interval measurement algorithm (Step 366) and the pulse arrival time measurement algorithm (Step 370). The peak detection result signal from the BCG module 26 is the second input to the RJ Interval algorithm (Step 366), while the peak detection result signal from the PPG module 26 is the second input to the pulse arrival time algorithm (Step 370). The processor 14 then calculates the blood pressure (Step 374) as the average of the blood pressure (bp1) calculated from the RJ Interval and the average of the blood pressure (bp2) calculated from the pulse arrival time.

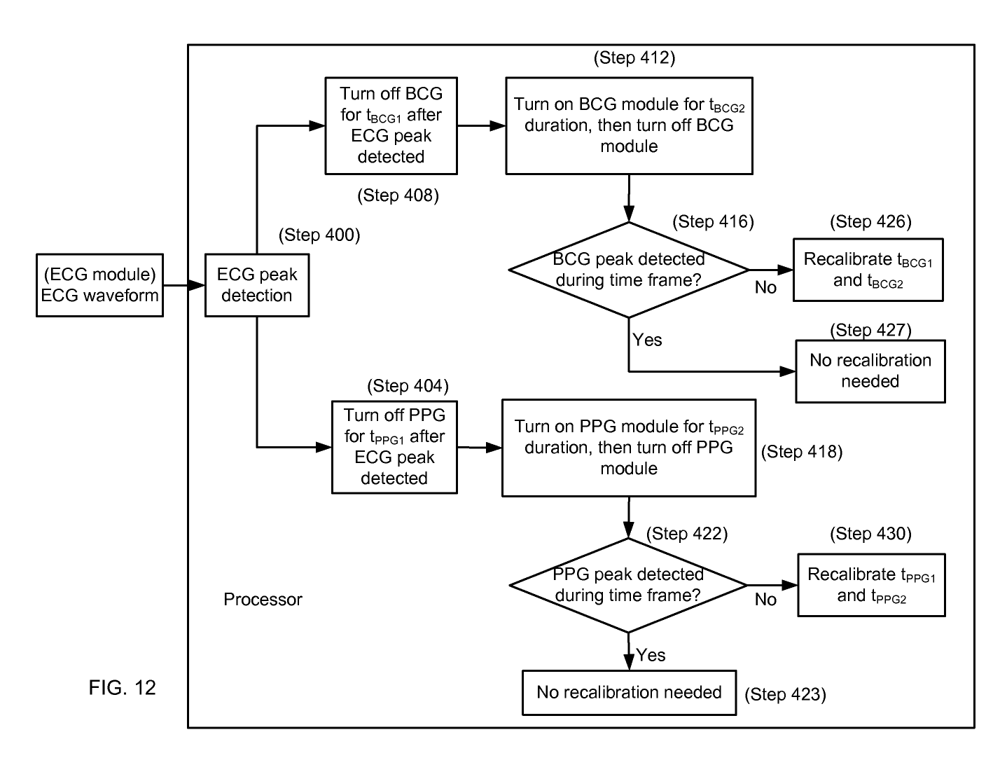

To reduce the amount of power consumed by the system, various modules may be turned off under various circumstances. In one embodiment (FIG. 12), an ECG waveform undergoes peak detection (Step 400). Once the peak is detected the BCG module is turned off or remains off if already off for a time period (t_{BCG1}) (Step 408). At the end of the time period (t_{BCG1}), the BCG module is turned on (Step 412) for a time period (t_{BCG2}), after which the BCG module is again turned off. If a peak is detected (Step 416) during the time period (t_{BCG2}), no recalibration is needed (Step 427) and the cycle repeats, saving power during the time the BCG module remains off. If, on the other hand, a peak in the BCG signal was not detected, then either the time period (t_{BCG1}) during which the BCG module was off was too long, or the time period (t_{BCG2}) during which the BCG module was on was too short. In either case, the two time periods are changed (Step 426) and the process repeats.

Similarly for the PPG module, once the peak is detected in the ECG, the PPG module is turned off or remains off if already off for a time period (t_{PPG1}) (Step 404). At the end of the time period (t_{PPG1}), the PPG module is turned on (Step 418) for a time period (t_{PPG2}), after which the PPG module is again turned off. If a peak is detected (Step 422) during the time period (t_{PPG2}), no recalibration is needed (Step 423) and the cycle repeats, saving power during the time the PPG module remains off. If, on the other hand, a peak in the PPG signal was not detected, then either the time period (t_{PPG1}) during which the PPG module was off was too long, or the time period (t_{PPG2}) during which the PPG module was on was too short. In either case, the two time periods are changed (Step 430) and the process repeats.

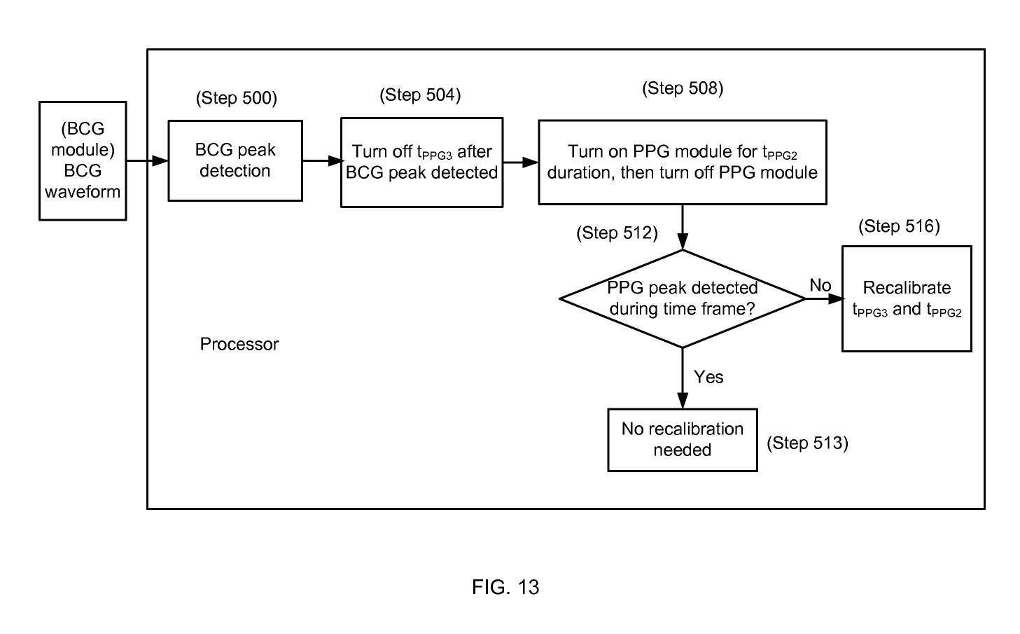

Referring to FIG. 13, if instead of the ECG signal a BCG signal is used to control the PPG module to conserve power, the procedure remains similar to the procedure just discussed. Once the peak is detected in the signal from the BCG module (Step 500), the PPG module is turned off or remains off if already off (Step 504) for a time period (t_{PPG3}). At the end of the time period (t_{PPG3}), the PPG module is turned on (Step 508) for a time period (t_{PPG2}), after which the PPG module is again turned off. If a peak is detected (Step 512) during the time period (t_{PPG2}), no recalibration is needed (Step 513) and the cycle repeats, saving power during the time the PPG module remains off. If, on the other hand, a peak in the PPG signal was not detected, then either the time period (t_{PPG3}) during which the PPG module was off was too long, or the time period (t_{PPG2}) during which the PPG module was on was too short. In either case, the two time periods are changed (Step 516) and the process repeats.

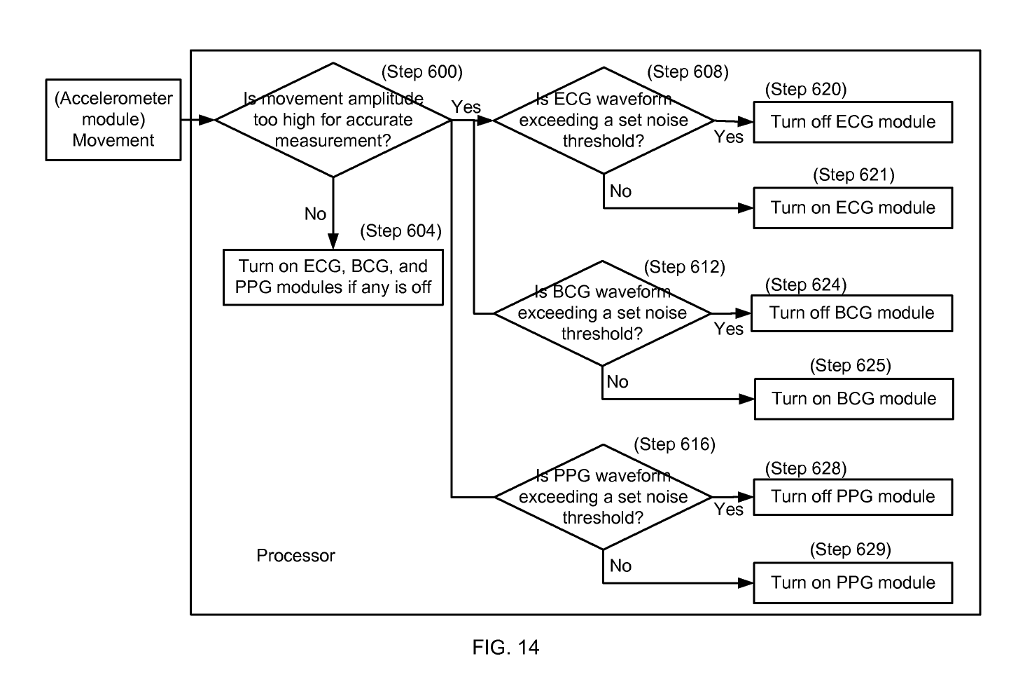

In a third embodiment, (FIG. 14) the system determines if the user's movements are too high to permit accurate measurement of vital signs. To do this, data from the accelerometer module 34 is examined to determine if the amplitude of patient movement is too high for accurate measurements to be made (Step 600). If such is not the case, then any of the ECG, BCG and PPG modules that are off is turned on (Step 604). At this time, the algorithm determines if the ECG waveform (Step 608), the BCG waveform (Step 612) and the PPG waveform (Step 616) exceed one or more predetermined noise thresholds. If this is the case for a given module, that module is turned off (Step 620, Step 624, Step 628). Otherwise, each of the ECG, BCG and PPG modules are turned on steps 621, 625 and 629 respectively.

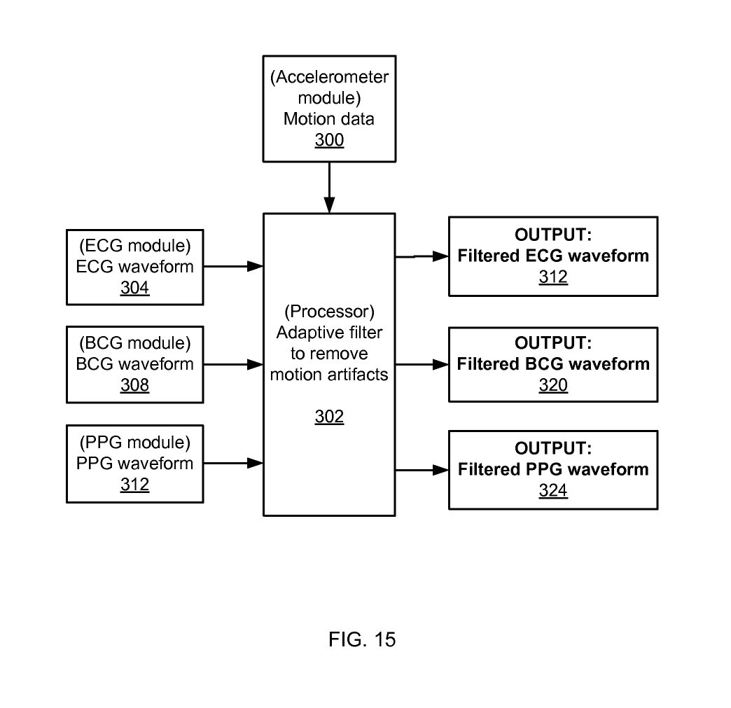

Referring to FIG. 15, motion data 300 from the accelerometer 34 can be used by the processor 14 to remove motion artifacts from the waveforms of the ECG module 304, the BCG module 308 and/or the PPG module 312 with an adaptive filter 302. The resulting corrected ECG 316, BCG 320 and PPG 324 waveforms are then used whenever a waveform is required by the calculation.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Furthermore, whereas particular embodiments of the invention have been described herein for the purpose of illustrating the invention and not for the purpose of limiting the same, it will be appreciated by those of ordinary skill in the art that numerous variations of the details, materials and arrangement of elements, steps, structures, and/or parts may be made within the principle and scope of the invention without departing from the invention as described in the claims.

Variations, modification, and other implementations of what is described herein will occur to those of ordinary skill in the art without departing from the spirit and scope of the invention as claimed. Accordingly, the invention is to be defined not by the preceding illustrative description, but instead by the spirit and scope of the following claims.

What is claimed is:

1. A vital signs monitor for wearing adjacent the ear, the monitor comprising:
a housing sized and shaped for fitting adjacent the ear of a wearer; and
an electronic module for measuring vital signs, the electronic module for measuring vital signs located within the housing and comprising:
a plurality of vital signs sensing modules, said plurality comprising at least two of the modules selected from the group comprising: a ballistocardiographic (BCG) module, a photoplethysmographic (PPG) module, an accelerometer module, a temperature measurement module, and an electrocardiographic (ECG) module; and
a processor, in electrical communication with the plurality of vital signs sensing modules, the processor calculating additional vital signs in response to signals from the plurality of vital signs sensing modules

2. The monitor of claim 1 wherein the processor measures heart rate from the ECG, the BCG, or the PPG module.

3. The monitor of claim 1 wherein the processor measures respiratory rate from the ECG, the BCG, or the PPG module.

4. The monitor of claim 1 wherein the processor determines orientation and motion in response to a signal from the accelerometer module.

5. The monitor of claim 1 wherein the processor measures stroke volume in response to a signal from the BCG module.

6. The monitor of claim 1 wherein the processor derives cardiac output in response to a signal from the BCG module.

7. The monitor of claim 1 wherein the processor calculates blood pressure in response to signals from the ECG and the BCG modules.

8. The monitor of claim 1 wherein the processor calculates blood pressure in response to signals from the ECG and the PPG modules.

9. The monitor of claim 1 wherein the processor calculates blood oxygenation in response to signals from the PPG module.

10. The monitor of claim 1 wherein the processor measures temperature in response to a signal from the temperature measurement module.

11. The monitor of claim 1 wherein the electronic module further comprises a visual or audible display module for providing information to a user in response to measured and calculated vital signs.

12. The monitor of claim 11 wherein the user is a wearer.

13. The monitor of claim 11 wherein the display module provides information to the user in response to measured and calculated vital signs that are out of acceptable range.

14. The monitor of claim 1 wherein the electronic module further comprises a memory module for saving recorded data.

15. The monitor of claim 1 wherein the electronic module further comprises a wireless communication module for sending data to a base-station.

16. The monitor of claim 15 wherein the base-station provides feedback to a user in response to measured and calculated vital signs.

17. The monitor of claim 15 wherein the base-station provides information to a user in response to measured and calculated vital signs that are out of acceptable range.

18. The monitor of claim 15 wherein the base-station controls the operation of the electronic module based on measured and calculated vital signs.

19. The monitor of claim 1 wherein the processor calculates the heart's relative change in pre-ejection period in response to signals from the ECG and the BCG modules.

20. The monitor of claim 1 wherein the processor performs, in response to one or more of the ECG signal, the BCG signal, and the PPG signal, an error detection for one or more of the heart rate, the respiratory rate, and the blood pressure.

21. The monitor of claim 1 further comprising a switch, in control of the processor, for turning on and off the BCG and the PPG modules in response to the ECG data, to reduce power consumption.

22. The monitor of claim 1 further comprising a switch, in control of the processor, for turning on and off the PPG module in response to the BCG data, to reduce power consumption.

23. The monitor of claim 1 further comprising a switch, in control of the processor, for turning on and off the ECG, the BCG, or the PPG module in response to accelerometer data to reduce power consumption.

24. The monitor of claim 1 wherein the blood pressure is calculated using cross-correlation of either the ECG and the BCG signals, or the ECG and the PPG signals.

25. The monitor of claim 1 wherein the heart rate is calculated using cross-correlation of two of the ECG, the BCG, and the PPG signals.

26. A PPG monitoring device comprising:
a housing sized and shaped for fitting adjacent the ear of a wearer; and
a PPG module located within the housing and comprising:
two light sources of different wavelengths positioned to transmit light into the skin adjacent the ear of the wearer;
a photodiode positioned to receive light reflected from the skin adjacent the ear of the wearer; and
a first amplifier in communication with the photodiode and providing a first amplifier output signal.

27. The PPG monitoring device of claim 26 further comprising a demodulator circuit in communication with the first amplifier.

28. The PPG monitoring device of claim 26 further comprising third and fourth light sources having wavelengths differing from the other light sources.

29. The PPG monitoring device of claim 26 further comprising a high pass filter and a second amplifier wherein the first amplifier is in communication with the high pass filter and second amplifier.

30. The PPG monitoring device of claim 29 further comprising a sample and hold circuit in communication with the second amplifier.

31. The PPG monitoring device of claim 26 wherein a difference amplifier in communication with the first amplifier subtracts a DC component and provides an AC component sent to the second gain amplifier.

32. The PPG monitoring device of claims 26, further comprising a low pass filter in communication with first amplifier.

33. The PPG monitoring device of claims 29 wherein the high pass filter is implemented in software.

34. The PPG monitoring device of claim 26 further comprising two additional light sources of different wavelengths selected to monitor functional oxygenated blood.

35. The PPG monitoring device of claim 26 further comprising:
a bandpass filter in communication with the first amplifier;
a demodulator in communication with the bandpass filter; and
a lowpass filter in communication with the demodulator.

36. The PPG monitoring device of claim 26 wherein the filters are implemented in software.

37. A BCG monitoring device comprising:
a housing sized and shaped for fitting adjacent the ear of a wearer;
two capacitive electrodes positioned adjacent to the ear of a wearer to transduce mechanical movements into electrical signals and
a BCG module located within the housing and comprising
a differential signal amplifier having an output terminal and two input terminals, each input terminal in communication with a respective one of the capacitive electrodes; and
an analog-to-digital converter in communication with the output terminal of the differential signal amplifier.

38. The BCG monitoring device of claim 37 further comprising a third electrode positioned in the mastoid region of the head of a wearer to reduce common mode interference signals.

39. The BCG monitoring device of claim 37 further comprising a filter in communication with the output terminal of the differential signal amplifier to reduce interference signals.

40. The BCG monitoring device of claim 37 further comprising an additional layer of electric shielding covering the two capacitive electrodes so as to reduce interference signals.

41. The BCG monitoring device of claim 37 further comprising of an accelerometer that senses head movements.

42. An ECG monitoring device comprising:
a housing sized and shaped for fitting adjacent the ear of a wearer;
two dry or gel-based electrodes positioned adjacent the ear of a wearer to detect the wearer's ECG;
and
an ECG module located within the housing and comprising:
a differential signal amplifier having an output terminal and two input terminals, each input terminal in communication with a respective one of the dry or gel-based electrodes; and
an analog-to-digital converter in communication with the output terminal of the differential signal amplifier.

43. The ECG monitoring device of claim 42 further comprising a third electrode positioned in the mastoid region of the head of a wearer to reduce common-mode interference signals.

44. The ECG monitoring device of claim 42 further comprising a filter in communication with the output terminal of the differential amplifier to reduce interference signals.

45. A method for monitoring PPG of the user comprising:
positioning a housing sized and shaped for fitting adjacent the ear of a wearer; the housing comprising
at least two light sources;
at least one photodiode;
a first amplifier in communication with the at least one photodiode and providing an amplified output signal; and
an analog-to-digital converter in communication with the amplified output signal;
transmitting light from each of the light sources in an alternating manner to the skin of the mastoid region of the wearer;
receiving, by the photodiode, the light reflected from the skin, tissue and bone of the mastoid region of the head of a wearer;
amplifying, by the first amplifier, a signal generated by the photodiode in response to the light reflected from the skin, tissue and bone to generate an amplified output signal; and
filtering the amplified output signal to reduce interference.

46. The PPG method of claim 45 wherein the signal filtering is performed in software.

47. A method for monitoring BCG, the method comprising:
positioning two capacitive electrodes in the mastoid region of the head of the wearer to sense head movements by transducing mechanical movements into electrical signals,
positioning a housing sized and shaped for fitting adjacent the ear of a user, the housing comprising a differential signal amplifier having an output terminal and two input terminals, each input terminal in electrical communication with a respective one of the two capacitive electrodes, and the output terminal in communication with an analog-to-digital converter.

48. The BCG method of claim 47 further comprising the step of reducing common-mode interference signals by placing a dry electrode in the mastoid region of the head of a wearer.

49. The BCG method of claim 47 further comprising filtering the output signal of the differential amplifier to reduce interference signals.

50. A method for monitoring BCG, the method comprising:
positioning a housing sized and shaped for fitting adjacent the ear of a user, the housing containing an accelerometer that senses head movements; and
sensing the movement of the head of the user.

51. The BCG method of claim 50 further comprising filtering the output of the accelerometer to reduce interference signals.

52. A method for monitoring an ECG, the method comprising:
positioning two electrodes in the mastoid region of the head of a wearer
positioning a housing sized and shaped for fitting adjacent the ear of a user, the housing containing:
a signal amplifier having two input terminals each in communication with a respective one of the electrodes, the amplifier having an output terminal; and
an analog-to-digital converters in communication with the output of the amplifier.

53. The ECG method of claim 52 further comprising positioning a third electrode in the mastoid region of the head of a wearer and using the third electrode to reduce common-mode interference signals.

54. The ECG method of claim 52 further comprising filtering the output of the differential amplifier to reduce interference signals.

55. The method of claim 54 wherein motion artifacts in the one or more of the ECG signal, the BCG signal, and the PPG signal, are corrected using motion data from the accelerometer module.
